(19) United States
(12) Patent Application Publication
(10) Pub. No.: US 2026/0131055 A1
(43) Pub. Date: May 14, 2026

(54) OPHTHALMIC VISCOELASTIC DEVICE

(71) Applicant: Carl Zeiss Meditec AG, Jena (DE)

(72) Inventors: Thorben Badur, Berlin (DE); Michael Thaller, Berlin (DE)

(21) Appl. No.: 19/435,247

(22) Filed: Dec. 29, 2025

Related U.S. Application Data
(63) Continuation of application No. PCT/EP2024/067685, filed on Jun. 24, 2024.

(30) Foreign Application Priority Data
Jun. 27, 2023 (DE) 10 2023 116 906.8

(51) Int. Cl. A61L 31/04; A61L 31/14; A61L 31/16; C08L 5/08

(52) U.S. Cl. CPC A61L 31/042; A61L 31/143; A61L 31/16; C08L 5/08

(57) ABSTRACT

An ophthalmic viscoelastic device includes at least one viscoelastic polymer, wherein the at least one viscoelastic polymer is cleavable into polymer chains of lower molecular weight, wherein the at least one viscoelastic polymer includes at least two polymer chains bonded to one another via at least one thermally and/or photochemically cleavable group, and wherein the at least one thermally and/or photochemically cleavable group is selected from compounds that can be coupled by a [2+2] cycloaddition and/or by click chemistry.

### CROSS-REFERENCE TO RELATED APPLICATIONS

This application is a continuation application of international patent application PCT/EP2024/067685, filed Jun. 24, 2024, designating the United States and claiming priority from German application 10 2023 116 906.8, filed Jun. 27, 2023, and the entire content of both applications is incorporated herein by reference.

### TECHNICAL FIELD

The disclosure relates to degradable ophthalmic viscoelastic devices.

### BACKGROUND

Cataract is a common disease, especially in the elderly, in which the eye lens gradually becomes opaque. This clouding of the natural lens leads to a loss of visual acuity. In order to restore vision, a cataract operation is required. The standard method for removing the cloudy lens core to create a capsular bag for the inserting of an artificial intraocular lens (IOL) is called phacoemulsification, with a device that generates ultrasonic vibrations.

Immediately before phacoemulsification, the anterior chamber is generally filled with an ophthalmic viscoelastic device (OVD). The viscoelastic OVD is used as a surgical aid for protecting the intraocular tissue (for example, the corneal endothelium during phacoemulsification), as a spacer (for example for support of the anterior chamber) and for facilitating intraocular procedures, for example for performance of a controlled capsulorhexis. However, such OVDs are also used in other eye operations, for example corneal transplants or glaucoma operations.

OVDs are generally water-based solutions containing viscoelastic polymers such as hyaluronic acid (HA), chondroitin sulfate (CS), hydroxypropylmethylcellulose (HPMC) or mixtures thereof. The viscoelastic composition may differ by the molecular weight of the polysaccharide dissolved in the solution, by the concentration of the polysaccharide, and by the viscosity of the solution. The rheological properties are highly dependent on the concentration and molecular mass of the polymers.

Document U.S. Pat. No. 6,745,776 B2 discloses methods of reducing postoperative intraocular pressure (IOP) after surgical procedures such as cataract operations and cornea transplants. The document describes the simultaneous administration of hyaluronic acid and hyaluronidase to the eye, either before, during or after the operation. This application is designed to reduce a significant rise in intraocular pressure after the operation and hence to prevent any possible temporary or permanent loss of sight.

In general, a distinction is made between two types of OVD. Highly viscous, cohesive OVDs maintain the space and build up pressure. For example, they are used to dilate the pupil before the anterior capsular bag of the lens is opened (capsulorhexis). Cohesive OVDs are included of polymers of high molecular weight.

By contrast, less viscous, dispersive OVDs envelop and protect the tissue. One of the most important applications is the formation of an adherent polymer barrier with a layer thickness of about 100 μm to about 1 mm between the corneal endothelium of the anterior chamber. Dispersive OVDs contain polymer chains of lower molecular weight compared to cohesive OVDs.

Both types of OVD are typically injected at the start of the procedure. During lens fragmentation, they can be flushed out of the incision. For that reason, they are replenished before implantation of an intraocular lens, since the anterior chamber has to expand for this step. After surgery, the OVDs have to be completely removed again from the eye. Degradation and removal of the OVD via natural drainage routes would take too long. During this period, the patient would suffer from a greatly increased intraocular pressure. This is not only painful but can also lead to an elevated risk of developing glaucoma.

### SUMMARY

Provided are ophthalmic viscoelastic devices (OVDs) for use in the context of eye surgery that reduce the risk of an increase in intraocular pressure after eye surgery.

In a first embodiment, an ophthalmic viscoelastic device is provided, including at least one viscoelastic polymer cleavable into polymer chains of lower molecular weight. In one embodiment thereof, the viscoelastic polymer includes at least two polymer chains bonded to one another via at least one thermally and/or photochemically cleavable group. In certain embodiments, that is, the at least one viscoelastic polymer of the described ophthalmic viscoelastic devices (OVDs) include two or more shorter polymer chains that are each bonded to one another via at least one thermally and/or photochemically cleavable group, via which predetermined “breaking points” are implemented. In contrast to, for example, hydrolytically cleavable polymers, in an embodiment of the viscoelastic polymer of the OVDs described herein, under biological conditions in the capsular bag or in the patient's eye, the described OVDs are fundamentally stable, and are specifically fragmented only after an external thermal and/or photochemical stimulus. Moreover, the cleaving of the viscoelastic polymer into the shorter polymer chains in certain embodiments does not give rise to any pharmaceutically active fragments. Instead, the viscoelastic polymer is, in such embodiments, specifically cleaved by the thermal and/or photochemical stimulus into the shorter polymer or oligomer chains with correspondingly lower molecular weight and different rheological properties, which are then small enough to be quickly transported away by the body through the trabecular meshwork or Schlemm's canal and broken down. Since the OVDs described herein are thus dissolved or broken down in the eye and are quickly transported away by the body via natural drainage routes and disposed of, the described OVDs no longer need to be removed after the operation. This saves time for doctors and operating room staff and allows for a higher number of procedures in a given time. Finally, such an OVDs increase patient safety since they reduce the likelihood that intraocular pressure will rise after surgery or problems with the trabecular meshwork will occur. In the simplest configuration, cleavage is possible without additional injections or invasive manipulations, for example by irradiation with daylight or ambient light or with light of a certain wavelength or a certain wavelength range. In general, “a/an” in the context of this disclosure should be read as the indefinite article, that is, always as “at least one” in the absence of any express indication of the contrary. Conversely, “a/an” can also be understood to mean “just one”.

In a further embodiment, the at least one thermally and/or photochemically cleavable group is selected from compounds that are couplable or coupled via a [2+2] cycloaddition and/or by click chemistry. In other words, the individual polymer chains are, in some embodiments, covalently bonded to thermally and/or photochemically cleavable groups, which in turn are couplable or coupled to one another via a [2+2] cycloaddition and/or by click chemistry. The coupling and/or cleaving is in principle, in certain embodiments, also reversible. A [2+2] cycloaddition, also known as 1,2 cycloaddition, describes the photochemical process in which cyclobutane derivatives are formed from alkenyl groups having an activated double bond. Useful compounds for this process include, for example, ketenes, allenes, cinnamic acids, coumarins, and also fluoroethylene and chlorofluoroethylene. Stereochemistry can be predicted by applying the Woodward-Hoffmann rules based on orbital symmetry. There is a distinction between thermally allowed and photochemically allowed [2+2] cycloadditions. The thermal [2+2] cycloaddition is known to be performed in three different ways: concerted, radical or ionic. In contrast to the concerted reaction, orbital symmetry plays no role in radical or ionic processes. Most concerted [2+2] cycloadditions are photochemically allowed electrocyclic reactions and are described by the Woodward-Hoffmann rules. Click chemistry refers to a class of chemical reactions that feature a fast, efficient and specific reaction between two coreactants to form a new compound. The reactions are generally very selective, and the products can be obtained in high yield. Click chemistry is of particularly good suitability for the production of functionalized molecules and materials used in biotechnology and medicine. One example of a click chemistry reaction is the Cu(I)-catalyzed azide-alkyne cycloaddition (also known as “Huisgen cycloaddition”). This connects an azide group to an alkyne group using a catalyst and with release of nitrogen in a fast and specific reaction to form a 1,2,3-triazole ring. This reaction is very useful for synthesizing functional molecules and polymers, since the alkyne and azide groups can be introduced into a multitude of molecules. Click chemistry has been found to be very useful because it provides a simple, efficient and selective way to chemically join compounds to one another. The major benefit of click chemistry is that it is very efficient and selective and runs under mild conditions. A further important advantage of the click reaction is its bioorthogonality, which means that it is compatible with biological systems. The azide and alkyne groups do not occur in natural systems, and so the click reaction is a very useful method for binding molecules to polymers or oligomers such as hyaluronic acid (HA) and other viscoelastic polymers or to the corresponding formation blocks of such viscoelastic polymers.

In one embodiment, the polymer chains described herein have a molar mass between 70 kDa and 200 kDa, in another embodiment the molar mass is between 76 kDa and 190 kDa, in a further embodiment the viscoelastic polymer has an average molecular weight of at least 0.5 MDa, or at least 2.5 MDa. All the measures and properties mentioned, individually or in any combination, have the effect that the polymer chains or fragments thereof formed after the cleaving of the thermally and/or photochemically cleavable group are of sufficiently small size to pass particularly reliably and completely or at least essentially completely through the trabecular meshwork or through the pores of Schlemm's canal and can be removed from the eye.

Further benefits arise in that the at least one viscoelastic polymer includes, in certain embodiments, at least one formation block from the group of hyaluronic acid, alginate, chitosan, methylcellulose, hydroxypropylmethylcellulose (HPMC), chondroitin sulfate, collagen and gelatin. This includes all derivatives and salts of the polymers, such as hyaluronates, alginates, and the like. In the context of the present disclosure, a formation block means monomeric, oligomeric or polymeric structural elements or polymer chains of the viscoelastic polymer. In some embodiments, the cleavage cleaves the viscoelastic polymer back into these original formation blocks or into polymer chains which in turn consist of these formation blocks. In one embodiment, the described viscoelastic polymer, apart from the thermally and/or photochemically cleavable groups, consists exclusively of one of the formation blocks, that is, as a non-limiting example, exclusively of hyaluronic acid blocks that are crosslinked indirectly, that is, via spacers, crosslinkers or other derivatizations, or directly via the thermally and/or photochemically cleavable group and form the viscoelastic polymer. Conversely, in other embodiments, the viscoelastic polymer consists of two or more different formation blocks, that is, for example, of hyaluronic acid blocks and HPMC blocks, etc.

In a further advantageous embodiment, the at least one viscoelastic polymer includes polymer chains bonded to one another end-to-end and/or via side chain positions via the at least one thermally and/or photochemically cleavable group. In other words, the viscoelastic polymer is formed from two or more shorter polymer chains, where the individual polymer chains are crosslinked with one another in a linear manner via respective terminal thermally and/or photochemically cleavable groups. This allows the size of the polymer chains to be predetermined particularly precisely after the cleavage of the cleavable groups, which enables particularly reliable transportation of the polymer fragments away via the aqueous humor and the trabecular meshwork. Conversely, the molar mass of the viscoelastic polymer is easily adjustable, and particularly long polymer chains are also producible, such that viscoelastic polymers with unusually high molar mass are also easily obtainable. Alternatively or additionally, the polymer chains described herein are, in some embodiments, joined to one another via side chain positions. That is, in certain embodiments, the polymer chains have one or more non-terminal thermally and/or photochemically cleavable groups, via which crosslinking with one or more other polymer chains is accomplished. A non-terminal side chain position in a polymer chain in such embodiments is also referred to as an “internal position” or “internal monomer.” These are positions between the ends of the chain other than the end groups of the polymer. The end groups are the outermost monomers at each end of the chain. The internal monomers generally have higher mobility than the end groups and therefore play an important role in the dynamics and properties of the polymer. Furthermore, this forms a three-dimensional network, whereby the rheological properties of the viscoelastic polymer is also adjustable in addition to degradability. In some embodiments, the individual polymer chains are additionally crosslinked via other functional groups, provided that degradability and outward transportability after the cleavage of the thermally and/or photochemically labile groups is assured to a sufficient degree. In this way, the viscoelastic properties of the viscoelastic polymer is likewise precisely adjustable.

In a further advantageous configuration, the ophthalmic viscoelastic device takes the form of a cohesive or dispersive ophthalmic viscoelastic device. A cohesive OVD is useful, for example, during eye surgery to fill the space between the lens and cornea and stabilize intraocular pressure, and offers several benefits that contribute to high safety and effectiveness of eye surgery. Owing to the degradability of the described OVDs, it is in some embodiments unnecessary to remove the OVD from the eye after eye surgery. In addition, it is possible in accordance with certain embodiments to produce viscoelastic polymers with particularly high average molecular weight, which allows for particularly good maintenance of space to be achieved. A dispersive OVD, in certain embodiments, particularly reliably produces an adherent polymer barrier during eye surgery. In one embodiment, the viscoelastic polymer in this case has a molecular weight of at most 2 MDa, in another embodiment, about 1 MDa, which in the case of hyaluronic acid corresponds to an average of about 1250 monomers. Furthermore, under standard conditions (25° C., 1 bar), the zero shear viscosity is not more than 100 Pas, or not more than 50 Pas. It will be apparent that the definition of standard conditions does not rule out the possibility that the OVDs described herein or their ingredients possess the specified properties at different temperatures and/or pressures as well.

Alternatively or additionally, a concentration of the at least one viscoelastic polymer based on the total volume of the ophthalmic viscoelastic device is between 0.1 mg/ml and 50 mg/ml. This also allows the properties of the OVD to be optimized to the respective end use.

Alternatively or additionally, the ophthalmic viscoelastic device described herein includes at least one therapeutic agent. In some embodiments, the therapeutic agent is an analgesic and/or an antioxidant. In certain embodiments the therapeutic agent is covalently bonded to the at least one viscoelastic polymer via at least one thermally and/or photochemically cleavable group and/or is embedded in the viscoelastic polymer. Such embodiments allow for a controlled delivery of the therapeutic agent, which is in some embodiments controllable by the degradation rate of the viscoelastic polymer, which in some embodiments additionally facilitates the eye operation. The therapeutic agent is in some embodiments covalently bonded to the at least one viscoelastic polymer, embedded in the polymer, or dissolved in the OVD. In embodiments of covalent bonding, the therapeutic agent likewise is attached via the same thermally and/or photochemically cleavable group as the polymer chains and is released without additional measures with the aid of the same thermal and/or photochemical stimulus. Alternatively, in some embodiments, the therapeutic agent is attached with the aid of a different group and accordingly released via a different stimulus. This allows controlled release of the therapeutic agent without cleaving the viscoelastic polymer.

Further benefits of the described OVDs arise when the described OVDs include a stabilizing agent, such as, for example, a free-radical scavenger, and/or magnetic particles, for example microparticles and/or nanoparticles. With the aid of a stabilizing agent, premature unwanted breakdown of the viscoelastic polymer is in some embodiments reliably prevented and providing stable storage of the OVD. Alternatively or additionally, the viscoelastic polymer is in some embodiments broken down or degraded by adding magnetic particles, such as, for example, microparticles and/or nanoparticles, to the OVD and subjecting them to an external magnetic field for breakdown of the viscoelastic polymer, which is referred to as magnetic field-assisted degradation. The basic idea is that the magnetic particles, which generally consist of magnetite or other ferromagnetic material, are mixed with the viscoelastic polymer, and then an external magnetic field is applied to the mixture. In such embodiments, without wishing to be bound by any particular theory, it is thought that the magnetic field induces an alternating current in the microspheres, which generates heat through magnetic hysteresis. The heat generated by the magnetic microspheres then causes the surrounding viscoelastic polymer to be thermally cleaved and degraded to the polymer chains. Alternatively, the heat generated by the magnetic particles also accelerates the photochemical degradation of the viscoelastic polymer by increasing the speed of chemical reactions. The specific conditions required for magnetic field-assisted degradation depend on the type of viscoelastic polymer used, the size and concentration of the magnetic particles, and the strength and frequency of the magnetic field. In general, the magnetic field has to be strong enough to induce an alternating current in the magnetic particles, but in some embodiments it is not so strong that the magnetic particles agglomerate and form lumps that could prevent uniform degradation of the viscoelastic polymer.

In a further advantageous embodiment, the ophthalmic viscoelastic device is stored in a thermos vessel and/or in an opaque vessel. In such embodiments, the shelf life of the described OVDs is increased and premature or unwanted breakdown is avoided.

Also disclosed is a cleaving device including means of cleaving at least one thermally and/or photochemically cleavable group of an ophthalmic viscoelastic device. With the aid of such a cleaving device, a thermal and/or photochemical stimulus is generated, which leads to the cleaving of the labile groups of the viscoelastic polymer of the OVD, and so this breaks down to the corresponding shorter polymer chains and is transported away from the patient's eye in a natural manner. Further features and benefits thereof are able to be inferred from the description provided hereinabove.

It may be the case that the cleaving device includes, as means, in one embodiment, a light source for generating light having a wavelength that photochemically cleaves the at least one group. In another embodiment, a light guide is provided that directs the light generated by the light source to the patient's eye. The cleaving device is positioned, in one embodiment, for example, on an operating microscope or integrated into an operating microscope and, on completion of the eye operation, exposes the patient's eye, over an area and/or at specific points or along an irradiation path, to light of a predetermined wavelength or a predetermined wavelength range, in order to initiate the photochemical breakdown of the group(s) of the viscoelastic polymer. Alternatively or additionally, the cleaving device takes the form of a contact lens, a pair of spectacles, or more generally of a device that covers at least some regions of the patient's eye. The light source provided is, in some embodiments, for example, an ring-shaped LED lamp. The patient then wears this cleaving device after the operation, for example during the recovery phase. The light generated by the cleaving device is then transmitted to the anterior chamber and cleaves the viscoelastic polymer of the OVD. In such an embodiment, this is accomplished without involvement of a surgeon and is convenient for the patient if the cleavage of the viscoelastic polymer takes longer than a few seconds. Alternatively or additionally, the cleaving device includes, as means, a magnetic device for generating a magnetic field, via which magnetic particles of the ophthalmic viscoelastic device are heatable in order to cleave the at least one group. Depending on the group used, it is split purely thermally thereby. Alternatively, magnetic heating is used to accelerate the reaction rate of a photochemical cleavage.

### BRIEF DESCRIPTION OF DRAWINGS

The invention will now be described with reference to the drawings wherein:

FIG. 1 provides a schematic diagram of the production steps for a viscoelastic polymer of an ophthalmic viscoelastic device of the invention in one working example;

FIG. 2 is a schematic diagram of the production steps for an alternative viscoelastic polymer of an ophthalmic viscoelastic device of the invention in a further working example;

FIG. 3 depicts a [2+2] cycloaddition reaction of coumarin-substituted polymer chains;

FIG. 4 shows a [2+2] cycloaddition reaction of cinnamic acid-substituted polymer chains;

FIG. 5 is a schematic diagram of a repeat disaccharide unit of hyaluronic acid;

FIG. 6 shows a diagram of a cleaving device in one working example; and,

FIG. 7 is a diagram of a cleaving device in a further working example.

### DETAILED DESCRIPTION

Ophthalmic viscoelastic devices (OVDs) are important aids for cataract surgery among other procedures. When the eye 26 (FIG. 6) is cut open, the aqueous humor drains from the eye 26. In order to facilitate cataract surgery, an OVD is inserted into the eye 26 to create space in the anterior chamber 24. OVDs suitable for creating space generally have relatively high viscosity (cohesive OVD type, viscosity >60 000 mPas). A further function of OVDs is the coating of endothelial cells, for which OVDs of lower viscosity are configured (dispersive OVD type, viscosity <60 000 mPas). Viscosity values are determinable under customary standard conditions for OVDs (25° C., 1 bar).

Both types of OVD are injected at the start of the procedure. During lens fragmentation, they can be flushed out of the incision. For that reason, they are replenished before implantation of an intraocular lens (IOL), since the anterior chamber has to expand for this step. After surgery, the OVDs have to be completely removed from the eye. The body cannot naturally drain conventional OVDs through the trabecular meshwork like the aqueous humor. If the OVDs remain in the eye after surgery, they block natural drainage from the eye, which can lead to increased intraocular pressure (IOP), which is very painful to patients and also entails the risk of glaucoma formation.

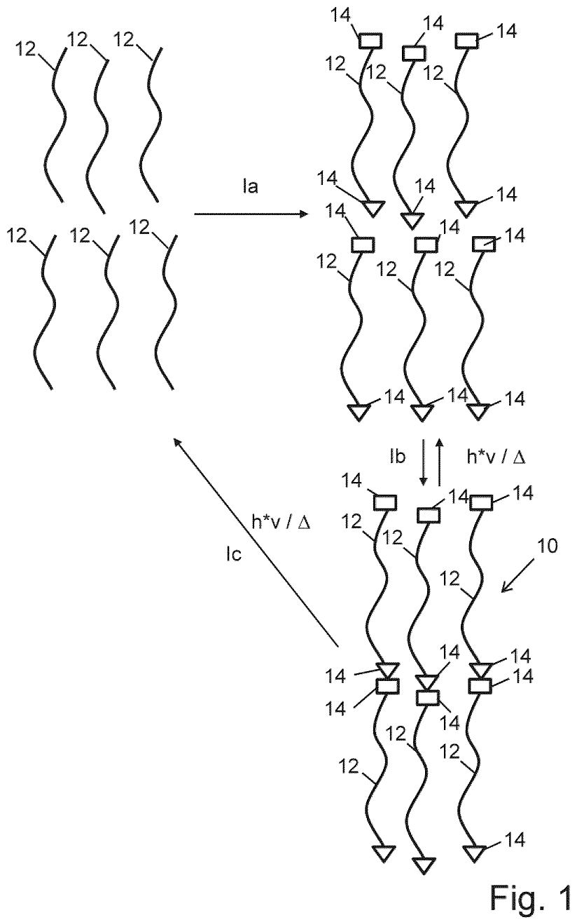

FIG. 1 shows a schematic diagram of the production steps for a viscoelastic polymer 10 of an ophthalmic viscoelastic device (OVD) of one working example. In this procedure, polymer chains 12 are first provided, each having a chain length that allows, in vivo, that is, in the patient's eye, rapid transportation away via natural drainage pathways such as the trabecular meshwork or Schlemm's canal without a significant increase in intraocular pressure (IOP). The polymer chains 12, which can also be referred to as formation blocks, in principle, in some embodiments, have the same or different lengths or (average) molar masses. In a step Ia, the polymer chains 12 are derivatized and provided with functional groups 14. In the working example shown, the functional groups 14 are each appended to the two ends of the individual polymer chains 12. In a step Ib, the functional groups 14 are then coupled end-to-end, whereby, depending on the reaction regime, viscoelastic polymers 10 of virtually any length are produced. For example, it is possible to produce viscoelastic substances with a particularly high molecular weight beyond 3 MDa, which were unobtainable to date via the established production routes. The functional groups 14 at the two ends of each polymer chain 12 generally differ or are of identical length, provided that they react with one another in the manner described. This generally means that either exclusively head-to-tail links are possible, or head-to-head, head-to-tail and tail-to-tail links. In the present working example, the head and tail groups 14 differ, such that only head-to-tail links are possible. Depending on the type of formation blocks 12 and functional groups 14, different chemical reaction pathways are possible. Reaction step Ib is in certain embodiments performed photochemically (h*v) or thermally (Δ) depending on the reaction type and is in some embodiments reversible, such that the polymer 10 is able to break down again to the individual polymer chains 12. In one embodiment, the break down is via photochemical reactions, which are in one embodiment thermally assisted. The viscoelastic polymer 10 is in some embodiments cleaved thermally and/or photochemically in some embodiments according to step Ic, wherein the functional groups 14 are additionally destroyed, modified or cleaved irreversibly, and so this step is not reversible. Both scenarios have benefits including in terms of production method and sensitivity to light exposure. In the case of photochemical cleavage, the required wavelength is in certain embodiments in a region that is blocked by the cornea, that is, below about 300 nm. This cleavage or activation wavelength is able to be varied by corresponding substituents on the molecular structure shown, for example, raised, to about 400 nm or more. Thus, neither the cornea nor the IOL would then be a barrier. This also applies, for example, to the compound shown in FIG. 4.

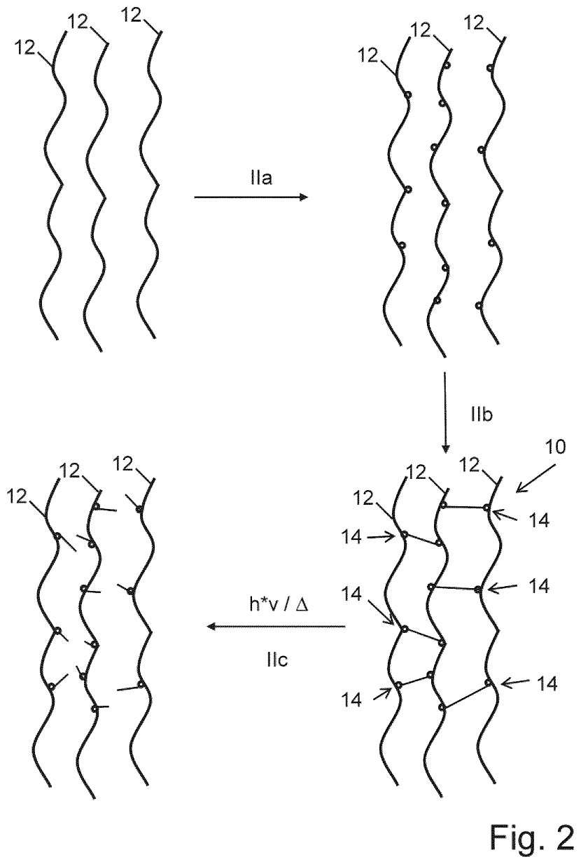

FIG. 2 shows a schematic diagram of the production steps for an alternative viscoelastic polymer 10 of an OVD described herein in a further working example. In contrast to the preceding working example, the polymer chains 12 are first chemically modified in an optional step IIa and crosslinked in step IIb. The crosslinking is not terminal or end-to-end, but via side chains of the polymer chains 12. This reaction is mainly concentration-dependent and is in certain embodiments controlled in such a way that the number of reactions or crosslinks per polymer strand of the viscoelastic polymer 10 is limited to 1, 2, or 3. In order to achieve chain growth greater than mere doubling of the molecular weight, at least two crosslinking sites should be provided per polymer chain 12, which are either in terminal position (FIG. 1), in a lateral position (FIG. 2), or in any combination thereof.

The concept described here is not limited to the use of a specific chemical group for implementation. A useful starting point may be structures suitable for [2+2] cycloaddition reactions, for example coumarin or cinnamic acid, since these compounds and reactions are known per se and are implementable reliably. In addition, there is a large amount of data on biocompatibility in the capsular bag, non-invasive initiation of the reaction by exposure to light, and chemical modification and alteration of the absorption maximum.

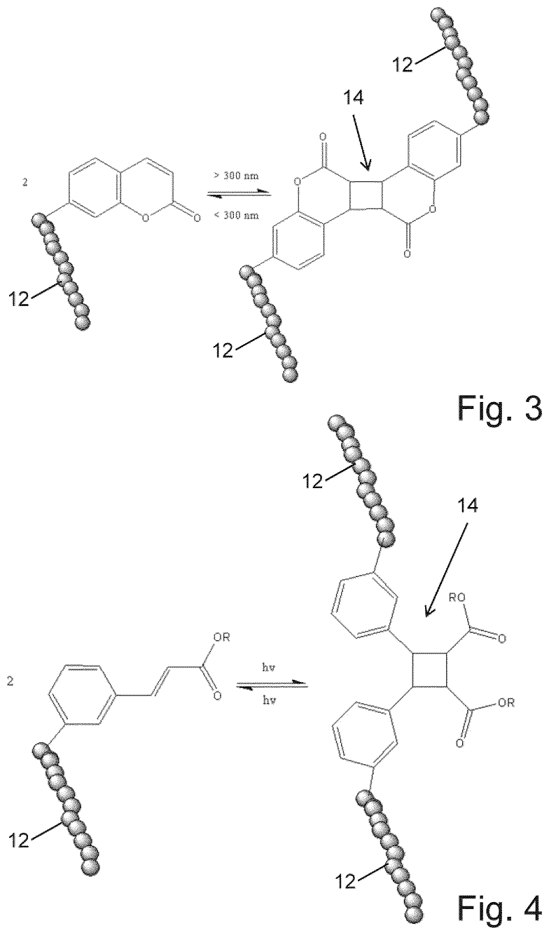

FIG. 3 shows, by way of example, a [2+2] cycloaddition reaction of coumarin-substituted polymer chains 12. Alternatively or additionally to the final modification of the polymer chains 12 shown, one or more coumarin groups are in certain embodiments provided as side groups of the polymer chains 12. Most concerted [2+2] cycloadditions are photochemically allowed electrocyclic reactions and are described by the Woodward-Hoffmann rules. Stereochemistry can be predicted by these rules. This is a [π2σ+π2σ] cycloaddition, with suprafacial ring closure of the orbitals. The reaction is initiated, for example, by irradiation with light having a wavelength of >300 nm, where the exact wavelength is able to be varied by derivatizations of the coumarin groups. The polymer chains 12 are then connected via the coumarin groups, which act as crosslinkers, which correspondingly increases their molecular weight. The resulting viscoelastic polymer 10 or the entire OVD with the viscoelastic polymer 10 should then be stored until use, for example, in brown glass vials or in fully opaque (thermal) containers, in order to reduce or completely avoid exposure to light and hence any risk of decay. After application to the patient's eye, the cyclobutane rings formed are then photochemically cleaved, in such embodiments, for example with light having a wavelength <300 nm, which causes the polymer 10 to break down again into its shorter polymer chains 12, which is then removed from the eye via natural drainage routes and degraded. In a particular embodiment, the absorption peak is tailored by the use of appropriate substituents, which allows wavelengths of up to 400 nm or more to be used for cleaving. This is useful in the case of an OVD that remains behind an intraocular lens (IOL) during surgery, since it can absorb either in the UV or even partly in the visible region (in the case of yellow IOLs).

As already mentioned, other chemical structures are contemplated herein to create these reversible bonds between polymer chains 12 and to create a viscoelastic having a viscosity that is alterable and adjustable as desired. One of these substance classes is that of cinnamates or cinnamic acid derivatives. In this regard, FIG. 4 shows a [2+2] cycloaddition reaction of cinnamic acid-substituted polymer chains 12. The general reaction principle corresponds to that of the coumarin groups discussed above. A wide range of other light-cleavable chemical groups are contemplated herein for this purpose, and coupled and split by the mechanism described. In order to covalently bind these functional groups to the polymer chains 12, it is possible to follow various reaction pathways known per se.

After the photocleavable viscoelastic polymers 10 have been produced, stabilizing compounds (for example, free-radical scavengers) are added to the OVD, in some embodiments, and the viscoelastic materials are then able to be packed in a suitable vessel for storage and transport with exclusion of light. In the case of subsequent administration by a surgeon, a distinction should be made between the required activation time, the reaction time for chemical cleavage, and the drainage time. Since intraocular pressure usually reaches its peak about 3 to 7 hours after surgery, a guideline for the reaction time is fixed in order to degrade the polymer 10 as quickly as possible after use and to distinctly reduce its viscosity for drainage in most instances within minutes or at least within a few hours. In certain embodiments, the reaction is complete or at least predominantly complete after 2 to 3 hours. As mentioned above, the activation time is, in some embodiments, set to a few seconds depending on the chemical structures chosen. In other embodiments, it takes up to a few hours, for example when daylight or ambient light is used to initiate the cleavage reaction.

The OVDs described herein, in certain embodiments, contain one or more therapeutic agents (for example, antibiotics), which are likewise released, for example, into the capsular bag when the polymer 10 is cleaved. The therapeutic agent(s) are, in some embodiments, embedded into or covalently bonded to the polymer 10. In the latter case, the covalent bonds are in certain embodiments accomplished with the same groups 14 as the crosslinking of the polymer chains 12, such that the therapeutic agent is released together with the cleavage of the polymer 10 or via the same mechanism and trigger as the cleavage of the polymer 10.

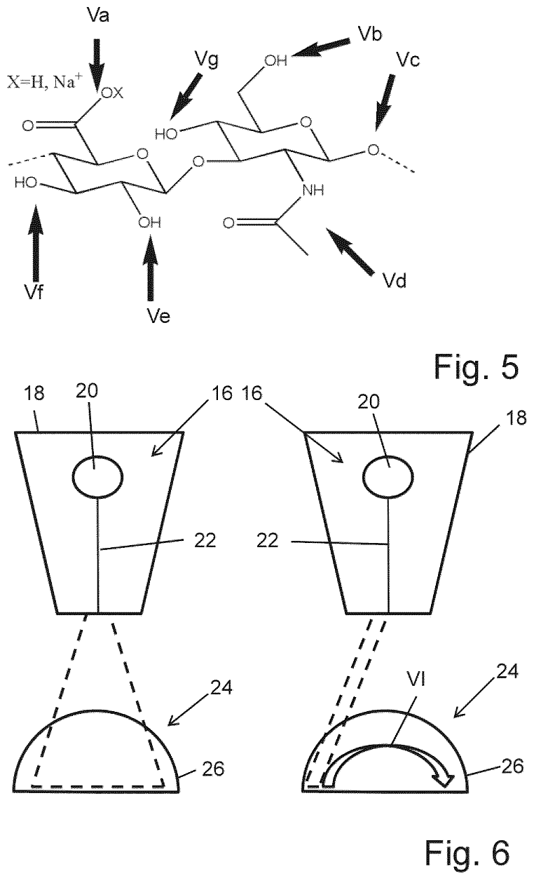

FIG. 5 shows a schematic diagram of a repeat disaccharide unit of hyaluronic acid, which can be used as formation block or as polymer chain 12 for the polymer 10. Such a D-glucuronic acid N-acetyl D-glucosamine disaccharide has a size of about 1 nm. Arrows Va-Vg are provided to mark various reactive functional groups and potential reaction sites for derivatization of hyaluronic acid (HA). Va indicates a carboxyl group, Vb a primary hydroxyl group, Vc the reductive end group of HA, Vd an N-acetyl group, and Ve, Vf, and Vg secondary hydroxyl groups. These groups Va-Vg are used, in certain embodiments, to attach functional groups 14 or crosslinkers to the HA framework in various ways. In addition to the modification of hyaluronic acid fordrug-releasing hydrogels, in certain embodiments, other chemical modifications are also known for a wide range of applications. With regard to the terminal binding of functional groups, ring-opening reactions or coupling reactions using the reducing end of hyaluronic acid are known. However, other viscoelastic polymers 10 or formation blocks or polymer chains 12 thereof are contemplated herein and able to be modified accordingly, which leads to a wide range of different contemplated application scenarios and options.

FIG. 6 shows a diagram of a cleaving device 16 in one working example. The cleaving device 16 in the present context is integrated into an operating microscope 18 (OPMI) and includes a light source 20, an optional light guide 22 and optionally a video camera (not shown). The viscoelastic polymer 10 is then cleaved, in this embodiment, either by full exposure of the anterior chamber 24 of the eye 26 (left-hand image) or by local scanning according to arrow VI along an irradiation path around the capsule in order not to overexpose the retina (right-hand image). Instead of light, the stimulus for cleaving is, in one embodiment, a magnetic field or another energy source. In other embodiments, the stimulus for cleaving is a thermal energy source.

Alternatively or additionally, the cleaving device 16 is configured to generate a magnetic field (not shown). It is possible thereby to cleave the polymer 10 of the OVD by additionally loading the OVD with magnetic micro-or nanospheres, which resonate with the magnetic field and thereby generate heat in the OVD (magnetic field-assisted degradation). The polymer 10 is then thermally cleaved in such embodiments. Alternatively, the heat generated is also used to support photochemical cleavage.

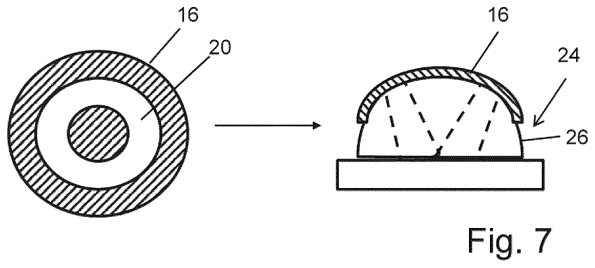

FIG. 7 shows a diagram of a cleaving device 16 in a further working example. The cleaving device 16 is generally configured as a device that partly or completely covers the eye 26, for example in the form of a contact lens, in the form of a pair of spectacles, or the like. The cleaving device 16 makes it possible to achieve a controlled long-term treatment environment. In such embodiments, the patient wears the individually adapted, for example, cleaving device 16 with an integrated light source 20 (for example, a ring-shaped LED, several LEDs or the like) in order to degrade the viscoelastic polymer 10 in the manner described above. The patient wears this cleaving device 16, for example, after the operation during the recovery phase. The light of a predetermined wavelength which serves for cleaving is then transferred into the anterior chamber 24 of the eye 26 and splits the viscoelastic polymer 10 into its short polymer chains 12. This proceeds, in some embodiments, without the involvement of a surgeon. In such embodiments, the cleaving device 16 is worn for as long as necessary for the substantial or complete degradation of the polymer 10.

The parameter values specified in the documents to define process and measurement conditions for the characterization of specific properties of the subject matter described herein should also be considered to be encompassed by the scope of the described invention in the context of variances for example owing to measurement errors, system errors, DIN tolerances, and the like.

It is understood that the foregoing description is that of the preferred embodiments of the invention and that various changes and modifications may be made thereto without departing from the spirit and scope of the invention as defined in the appended claims.

### LIST OF REFERENCE SIGNS

10 polymer
12 polymer chain
14 group
16 cleaving device
18 operating microscope
20 light source
22 light guide
24 anterior chamber
26 eye
Va-Vg functional group
VI exposure path

1. An ophthalmic viscoelastic device comprising at least one viscoelastic polymer,
wherein said at least one viscoelastic polymer is cleavable into polymer chains of lower molecular weight,
wherein the at least one viscoelastic polymer comprises at least two polymer chains bonded to one another via at least one thermally and/or photochemically cleavable group, and
wherein the at least one thermally and/or photochemically cleavable group is selected from compounds that can be coupled by a [2+2] cycloaddition and/or by click chemistry.

2. The ophthalmic viscoelastic device as claimed in claim 1,
wherein the at least two polymer chains have a molar mass between 70 kDa and 200 kDa, and/or
wherein the at least one viscoelastic polymer has an average molecular weight of at least 0.5 MDa.

3. The ophthalmic viscoelastic device as claimed in claim 1, wherein the at least one viscoelastic polymer further comprises at least one formation block selected from hyaluronic acid, alginate, chitosan, methylcellulose, hydroxypropylmethylcellulose, chondroitin sulfate, collagen, and gelatin.

4. The ophthalmic viscoelastic device as claimed in claim 1, wherein the at least one viscoelastic polymer further comprises polymer chains bonded to one another end-to-end and/or via side chain positions via the at least one thermally and/or photochemically cleavable group.

5. The ophthalmic viscoelastic device as claimed in claim 1,
wherein the viscoelastic device is a cohesive or dispersive ophthalmic viscoelastic device,
wherein a concentration of the at least one viscoelastolymer based on a total volume of the ophthalmic viscoelastic device is between 0.1 mg/ml and 50 mg/ml, and/or
wherein the viscoelastic device comprises at least one therapeutic agent.

6. The ophthalmic viscoelastic device as claimed in claim 1, further comprising a stabilizer.

7. The ophthalmic viscoelastic device as claimed in claim 1, wherein the ophthalmic viscoelastic device is stored in a thermos vessel and/or in an opaque vessel.

8. The ophthalmic viscoelastic device as claimed in claim 1, wherein the at least two polymer chains have a molar mass between 76 kDa and 190 kDa.

9. The ophthalmic viscoelastic device as claimed in claim 1, wherein the at least one viscoelastic polymer has an average molecular weight of at least 2.5 MDa.

10. The ophthalmic viscoelastic device as claimed in claim 5, wherein the at least one therapeutic agent is an analgesic and/or an antioxidant.

11. The ophthalmic viscoelastic device as claimed in claim 5, wherein the therapeutic agent is covalently bonded to the at least one viscoelastic polymer, and/or wherein the therapeutic agent is embedded in the viscoelastic polymer via the at least one thermally and/or photochemically cleavable group.

12. The ophthalmic viscoelastic device as claimed in claim 6, wherein the stabilizer is a free-radical scavenger and/or magnetic particles.

13. The ophthalmic viscoelastic device as claimed in claim 6, wherein the stabilizer comprises microparticles and/or nanoparticles.
